(19) United States
(12) Patent Application Publication
(10) Pub. No.: US 2002/0058252 A1
(43) Pub. Date: May 16, 2002

(54) Short shared nucleotide sequences

(76) Inventor: Evgueni V. Ananiev, Johnston, IA (US)

Correspondence Address: PIONEER HI-BRED INTERNATIONAL INC., 7100 N.W. 62ND AVENUE, P.O. BOX 1000, JOHNSTON, IA, 50131, US

(21) Appl. No.: 09/730,468

(22) Filed: Dec. 4, 2000

Related U.S. Application Data
(60) Provisional application No. 60/169,157, filed on Dec. 6, 1999.

(51) Int. Cl. C12Q 1/68; G06F 19/00; G01N 33/48; G01N 33/50

(52) U.S. Cl. 435/6; 702/20

(57) ABSTRACT

A method of selecting sets of short shared nucleotide sequences from amongst members of nucleic acid populations and identifying subsets of those selected short shared nucleotide sequences that differentiate those members from one another. Probes corresponding to the sets of short shared nucleotide sequences can be synthesized and utilized, inter alia, to detect target nucleic acids in a sample population, to provide expression profiles, or to identify polymorphisms of a gene. The invention also includes integrated systems for performing various steps involved in the method and certain probe compositions.

### CROSS-REFERENCES TO RELATED APPLICATIONS

This application is related to U.S. Ser. No. 60/169,157 “SHORT SHARED NUCLEOTIDE SEQUENCES” by Ananiev, filed Dec. 6, 1999, which is incorporated by reference herein in its entirety for all purposes. The present application claims priority to and the benefit of this related application pursuant to 35. U.S.C. § 119(e).

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to a method of selecting sets of short shared nucleotide sequences from amongst the members of nucleic acid populations and identifying subsets of those selected short shared nucleotide sequences that differentiate those members from one another. Probes corresponding to the sets of short shared nucleotide sequences can be synthesized and utilized, inter alia, to detect target nucleic acids in a sample population, to provide expression profiles, or to identify allelic variants of a gene. The invention also includes integrated systems for performing various steps involved in the methods and certain probe compositions.

### BACKGROUND OF THE INVENTION

Diverse hybridization technologies have been developed for use in, among other things, the identification of genes and the evaluation of their levels of expression across various tissue types, developmental stages, and physiological states. For example, incomplete and error-containing sequences from one or both ends of cDNA clones, i.e., expressed sequence tags (or ESTs), have been utilized for gene identification. Adams, M. et al., (1991) “Complementary DNA Sequence: Expressed Sequence Tags and Human Genome Project,” Science 252, 1651-1656; Wilcox. A. S. et al., (1991) “Use of 3′ Untranslated Sequences of Human cDNAs for Rapid Chromosome Assignment and Conversion to STSs: Implication of an Expression Map of the Genome,” Nucleic Acids Res. 13, 1827-1843; and Okubo, K. et al., (L992) “Large Scale cDNA Sequencing for Analysis of Quantitative and Qualitative Aspects of Gene Expression,” Nature Genet. 2, 173-178. To some extent, the generation of ESTs has also been an important strategy for the characterization of expression levels. Meier-Ewert, S. et al., (1998) “Comparative Gene Expression Profiling by Oligonucleotide Fingerprinting,” Nucleic Acids Res. 20, 2216-2223. However, the technique does have several inherent limitations, including a relatively high cost per sample, problems in correctly identifying internal sequence changes, and a difficulty in identifying motif sequences located outside the sequence stretches. Id.

Partial sequencing by hybridization of randomly selected cDNA clones using short oligonucleotide probes has been suggested as a technique for identifying active genes and for determining transcription levels by counting the occurrences of clones that represent the same gene in tissue-specific libraries. Drmanac, R. et al., (1991) “Partial Sequencing by Oligohybridization: Concept and Applications in Genome Analysis.” in Proceedings of the First International Conference on Electrophoresis, Supercomputing and the Human Genome, (C. Cantor and H. Lims, Eds.), 60-75, World Scientific, Singapore; Lennon, G. S. and Lehrach, H. (1991) “Hybridization Analysis of Arrayed cDNA Libraries,” Trends Genet. 7, 60-75; Drmanac, S. and Drmanac, R. (1994) “Processing of cDNA and Genomic Kilobase-Size Clones for Massive Screening, Mapping and Sequencing by Hybridization,” Biotechniques 17, 328-336; and Drmanac, R. et al., (1992) “Sequencing by Hybridization: Towards an Automated Sequencing of One Million M13 Clones Arrayed on Membranes,” Electrophoresis 13: 566-573. A distinguishing feature of this approach, relative to some other hybridization-based procedures, is the application of probes with lengths approaching those of restriction sites. Drmanac, S. et al., (1996) “Gene-Representing cDNA Clusters Defined by Hybridization of 57,419 Clones from Infant Brain Libraries with Short Oligonucleotide Probes,” Genomics 37, 29-40. This permits the detailed parallel analysis of randomly selected cDNA clones, usually 0.5 to 3 kilobases in length, by scoring as few as 100 probes. Id.

In one study, a partial sequencing by hybridization method, involving the hybridization of clones from both ordinary and normalized cDNA libraries with mainly 7-mer probes, was used to assess the diversity of genes expressed in the infant brain by identifying individual clones or clone clusters for approximately 20,000 distinct genes. Id. In this experiment, arrayed clones were hybridized to diverse sets of probes ranging from 200 to 320 in number. Drmanac, R. et al., (1994) “Requirements in Screening cDNA Libraries for New Genes and Solutions Offered by SBH Technology,” in Identification of Transcribed Sequences (U. Hochgeschwender and K. Gardiner, Eds.) 239-251, Plenum, N.Y. Approximately, a third of those probes were selected to be highly frequent 7-mers and 8-mers in coding regions, so that the majority of clones would give a scoreable signal. Drmanac, S. et al., (1996) Genomics 37, 29-40. Additionally, to improve discrimination for clones representing 3′ untranslated regions, a group of 7-mer probes were selected that were presumed to be frequent in these sequences or common in Alu repeats, totaling another 30%. Also, more than a quarter of the probes overlapped by all but one base with one of the already selected probes on one or both sides. These probes were intended to indicate the existence of extended segments of identical sequence between compared clones. This overall probe selection strategy enabled the discrimination of clones with a small probe set. Id.

In another study, 29,570 clones in duplicate from both original and normalized infant brain cDNA libraries were hybridized with 107-215 randomly chosen 7-mer oligonucleotide probes to obtain oligonucleotide sequence signatures. Milosavljevic, A. et al., (1996) “Discovering Distinct Genes Represented ii 29,570 Clones from Infant Brain cDNA Libraries by Applying Sequencing by Hybridization Methodology,” Genome Res. 6, 132-141. The oligonucleotide sequence signatures were compared and grouped based on mutual similarity into 16,741 clusters, each corresponding to a distinct cDNA. Id. Additionally, a number of distinct cDNAs were identified by matching their 107-probe oligonucleotide sequence signatures against GenBank® entries. Id.

In a similar approach, others have advanced techniques involving the hybridization of short oligonucleotide probes under high stringency conditions to derive sequence dependent “fingerprints.” Meier-Ewert, S. et al., (1998) “Comparative Gene Expression Profiling by Oligonucleotide Fingerprinting,” Nucleic Acids Res. 20, 2216-2223. As with the other methods, these fingerprints can identify genes and assess their levels of expression in different tissues. Id. Additionally, various automated procedures to facilitate large-scale cDNA analysis by oligonucleotide hybridization to arrayed clone libraries immobilized on nylon membranes can be utilized with this technique. Meier-Ewert, S. et al., (1993) Nature, 361, 375-376 and Meier-Ewert, S. et al., (1994) J. Biotechnol. 35, 191-203. The oligonucleotide probes were designed as 10-mers with a common 8-mer core (i.e., NXXXXXXXXN). These probes were used, since the stability of 10-mer duplexes is significantly greater and therefore easier to detect. Furthermore, for any given hybridization signal it was not possible to determine which individual 10-mer probe hybridized to the target, the sequence information for each signal was limited to the eight nucleotide core common to each probe. Id.

Unlike the hybridization techniques discussed above, the present invention relates to a method of selecting short shared nucleotide sequences whose sequences are known and then generating probes based upon those sequences for use in various hybridization applications.

### SUMMARY OF THE INVENTION

The present invention provides methods of selecting and identifying differentiating subsets of short shared nucleotide sequences. The methods include selecting a set of short shared nucleotide sequences from amongst members, e.g., genes, of a nucleic acid population. Each short shared nucleotide sequence can correspond to a nucleic acid subsequence that is common to two or more members of the nucleic acid population. Alternatively, short shared nucleotide sequences can be further limited to correspond to the coding regions of at least two members, e.g., exons in eukaryotic genes. This limitation can, e.g., facilitate expression profiling applications. Once the set is selected, differentiating subsets of the short shared nucleotide sequences can be identified. A differentiating subset includes a subset of the selected short shared nucleotide sequences which differentiates an individual member of the nucleic acid population from other members of the nucleic acid population.

Based upon the identified differentiating subsets of sequences/probes, individual target nucleic acid sequences can be distinguished from other members of a sample population by detecting the differentiating subsets (e.g., by probe detection) that correspond to those target sequences. This can be accomplished by providing, e.g., synthesizing (e.g., in an automated nucleic acid synthesizer), a set of differentiating nucleic acid probes corresponding to the selected set of short shared nucleotide sequences, and then hybridizing and detecting the hybridization of at least one of those probes to the members of the population. This cycle of hybridizing and detecting steps can be repeated until a plurality of the differentiating nucleic acid probes have been hybridized to the nucleic acid population and those hybridizations detected. Target nucleic acids can then be distinguished by determining the sets of differentiating nucleic acid probes that hybridized to them. This method can be expanded to determine the sets of differentiating nucleic acid probes that hybridized to each member of a nucleic acid population. Additionally, at least one of the steps included in the method of the present invention can occur in vitro or in silico.

In one embodiment of the methods of the present invention, the hybridizing step additionally includes concomitantly hybridizing at least one competitor differentiating nucleic acid probe to at least one differentiating nucleic acid probe. A competitor differentiating nucleic acid probe can be complementary to at least one of the differentiating nucleic acid probes in the hybridization mixture. This embodiment is designed to minimize non-specific cross-hybridization.

In another embodiment of the invention, a target nucleic acid sequence, e.g., a cDNA, can be detected at least twice by identifying members of a nucleic acid population that hybridize to the same set of differentiating nucleic acid probes. Among other things, this can help to minimize resequencing when directly prepared cDNA libraries are used.

In an additional embodiment of the present invention, the set of short shared nucleotide sequences can be selected from at least one nucleic acid sequence database, e.g., an expressed sequence tag database. In addition, the short shared nucleotide sequences can correspond to nucleic acid sequences selected from one or more, e.g., restriction sites, homopolymers, and/or sequence repeats. In another embodiment, the set of short shared nucleotide sequences includes a set of degenerate oligonucleotides. Each degenerate oligonucleotide includes at least one region of sequence identity and at least one region of sequence heterogeneity as compared to the other degenerate oligonucleotides of the set of degenerate oligonucleotides.

The nucleic acid populations involved in the methods of the invention can include RNAs, DNAs and/or cDNAs. Furthermore, those populations can be derived from any organism; in certain preferred embodiments they are derived from plants. In the various embodiments of the present invention, members of nucleic acid populations can include one or more, e.g., expressed sequence tags, promoters, enhancers, exons, introns, domains, genes, polymorphisms, operons, gene clusters, gene families, and/or cloned nucleic acids. Additionally, samples including members of nucleic acid populations can include standardized or non-standardized concentrations of each member.

The methods of the present invention include differentiating nucleic acid probes that can be polynucleotides of various lengths, e.g., 6, 8, 10, 12, 15 or more nucleotides. Additionally, those differentiating nucleic acid probes can be synthesized in an automated nucleic acid synthesizer.

In certain embodiments of the invention, the members of sample nucleic acid populations or the set of differentiating nucleic acid probes can be present in an array of nucleic acids. Alternatively, the members of sample nucleic acid populations or the set of differentiating nucleic acid probes can be attached, e.g., covalently or non-covalently, to a solid support, e.g., paper, nitrocellulose, nylon, controlled pore glass, plastic, etc., and used, inter alia, to provide a short shared nucleotide sequence hybridization pattern or other profile of, e.g., an attached nucleic acid sequence member.

Another embodiment of the invention includes detecting polymorphisms in members of nucleic acid populations. This can be accomplished by detecting the hybridization of at least one differentiating nucleic acid probe to an individual member, e.g., a gene or a quantitative trait loci (QTL), in addition to the hybridization of the differentiating subset of probes that corresponds to the individual member. Also, the nucleic acid population can correspond to a multigene family and detected polymorphisms can map a member within a nucleic acid population.

The present invention also includes an integrated system that includes a computer or computer readable medium that includes a database with at least one sequence record that includes a plurality of non-homologous character strings corresponding to at least one nucleic acid population and at least one derivative sequence record that can include at least one set of short shared nucleotide sequences. The system also includes a user input interface that allows the user to selectively view the sequence record. Additionally, the system can include a sequence search and selection instruction set that searches the plurality of non-homologous character strings corresponding to the members of the nucleic acid population and selects one or more subsequences common to at least two of the plurality of non-homologous character strings.

The integrated system of the invention optionally includes an automated nucleic acid synthesizer coupled to an output of the computer or computer readable medium. The automatic synthesizer can accept instructions from the computer or computer readable medium and those instructions can direct the synthesis of differentiating nucleic acid probes which correspond to the one or more subsequences common to the at least two of the plurality of non-homologous character strings.

The system can further include one or more robotic or microfluidic control elements for manipulating at least one set of differentiating nucleic acid probes or the members of a nucleic acid population. The manipulations can include selecting the set of differentiating nucleic acid probes or the members of the nucleic acid population, reverse-transcribing RNAs, and synthesizing the set of differentiating nucleic acid probes. Other manipulations can include amplifying (and purifying amplified) members of a nucleic acid population, arraying the set of differentiating nucleic acid probes or the members of the nucleic acid population, and hybridizing the set of differentiating nucleic acid probes to the members of the nucleic acid population. The control elements can also be used for flowing the members of a nucleic acid population through at least one channel in a microfluidic device exposed sequentially to at least one labeled set of differentiating nucleic acid probes.

The integrated system can also include a detector for detecting hybridization patterns corresponding to target nucleic acid sequences. The system can additionally include a user readable output element that displays the subsequences common to at least two of the plurality of non-homologous character strings produced by the sequence search and selection instruction set. A user readable output element can also display hybridization patterns that correspond to target nucleic acid sequences.

The computer or computer readable medium of the integrated system can additionally include an instruction set for reverse-transcribing RNA sequences, selected from members of the nucleic acid population, into cDNA sequences. This embodiment can further include a search and selection instruction set that searches the plurality of non-homologous character strings corresponding to the members of a nucleic acid population and selects one or more subsequences common to at least two of the plurality of non-homologous character strings. The integrated system of the present invention can also include a user readable output element that displays short shared nucleotide sequences produced by the search and selection instruction set.

The present invention also includes a composition that includes one or more libraries of differentiating nucleic acid probes that correspond to the set of short shared nucleotide sequences. The sets of short shared nucleotide sequences collectively include a plurality of differentiating nucleic acid probe member types, in which differentiating subsets of the plurality of probe member types differentiate individual members of a nucleic acid population from each other. Similarly, compositions that include one or more libraries of competitor differentiating nucleic acid probes that correspond to a set of nucleic acid sequences that are complementary to a set of short shared nucleotide sequences are included.

Definitions

Unless otherwise indicated, the following definitions supplement those in the art.

An “array” is a spatially defined pattern of nucleic acid sequences, e.g., short shared nucleotide sequences or members of a nucleic acid population, typically on a solid support. A “preselected array of nucleic acid sequences” is an array of spatially defined nucleic acids typically on a solid support which is designed before being constructed (i.e., the arrangement of at least some of the polymers on the solid substrate during synthesis is deliberate, and not random).

The phrase “computer system” or “integrated system” in the context of this invention refers to a system in which data entering a computer corresponds to physical objects or processes external to the computer, e.g., nucleic acid hybridization or protein binding data and a process that, within a computer, causes a physical transformation of the input signals to different output signals. For example, the input data, e.g., hybridization of expression products on a specific array, is transformed to output data, e.g., the identification or counting of the sequence hybridized, comparison to similar arrays with different test materials, counting and categorization of expression products or the like. The process within the computer can include a program or instruction set by which positive (or negative) hybridization signals are recognized by the computer system and attributed to a region of an array, or other expression profile format (e.g., simple counting of array signals). The program can determine which region of the array the hybridized expression products are located on and, optionally, the specific corresponding sequences which the probe is based on (as noted above, no sequence information is required for making or assessing expression profiles).

Two nucleic acids “correspond” when they have the same sequence, or when one nucleic acid is complementary to the other, or when one nucleic acid is a subsequence of the other, or when one sequence is derived, by natural or artificial manipulation from the other.

“Differentiating subsets” are subsets of a set of short shared nucleotide sequences that are unique to individual members of a nucleic acid population. For example, in the context of a hybridization experiment, a probe set corresponding to the differentiating subset allows one member of the nucleic acid population to be distinguished from any other member. A set of “differentiating nucleic acid probes” is a set of probes that can, e.g., correspond to a differentiating subset of a set of short shared nucleotide sequences, or a set of short shared nucleotide sequences. A “competitor differentiating nucleic acid probe” is a probe that is complementary to or substantially complementary to a differentiating nucleic acid probe.

A nucleic acid “domain” is a discrete nucleic acid region or subsequence. It can be conserved or not conserved between a plurality of homologous nucleic acids. Generally, a domain is specified by comparing two or more sequences, where a region of sequence diversity between sequences constitutes a “sequence diversity domain,” while a region of similarity is a “sequence similarity domain.”

An “expression profile” is the result of detecting a representative sample of expression products from a cell, tissue or whole organism, or a representation (picture, graph, data table, database, etc.) thereof. For example, many RNA expression products of a cell or tissue can simultaneously be detected on a nucleic acid array, or by the technique of differential display or modification thereof such as Curagen's “GeneCalling™” technology. Similarly, protein expression products can be tested by various protein detection methods, such as hybridization to peptide or antibody arrays, or by screening phage display libraries or by mass spectrometric approaches (see e.g., Hutchens et al., U.S. Pat. 5,719,060). A “portion” or “subportion” of an expression profile, or a “partial profile” is a subset of the data provided by the complete profile, such as the information provided by a subset of the total number of detected expression products.

Nucleic acids “hybridize” when they associate, typically in solution (or with one component fixed to a solid support). Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays,” (Elsevier, N.Y.), as well as Current Protocols in Molecular Biology, F. M. Ausubel et al., eds., Current Protocols, a joint venture between Greene Publishing Associates, Inc. and John Wiley & Sons, Inc., (1999 Supplement). Hames and Higgins (1995) Gene Probes 1 IRL Press at Oxford University Press, Oxford, England, and Hames and Higgins (1995) Gene Probes 2 IRL Press at Oxford University Press, Oxford, England provide details on the synthesis, labeling, detection and quantification of DNA and RNA, including oligonucleotides.

The term “identical,” in the context of two or more nucleic acid sequences, refers to two or more sequences or subsequences that are the same or have a specified percentage of nucleotides that are the same, when compared and aligned for maximum correspondence, as measured, e.g., using an alignment instruction set or by visual inspection. The phrase “substantially identical,” in the context of two nucleic acids refers to two or more sequences or subsequences that have at least about 50%, preferably about 80%, most preferably about 90-98% or more nucleotide identity, when compared and aligned for maximum correspondence, as measured using an alignment instruction set or by visual inspection.

The term “label” refers to a composition detectable by spectroscopic, photochemical, biochemical, immunochemical, or chemical means. For example, useful nucleic acid labels include ³²P, ³⁵S, fluorescent dyes, electron-dense reagents, enzymes (e.g., as commonly used in an ELISA), biotin, dioxigenin, or haptens and proteins for which antisera or monoclonal antibodies are available.

A “library” is a set of nucleic acid or polypeptide sequences. The set can be pooled, or can be individually accessible. The nucleic acid sequences can comprise DNA, RNA, or cDNA.

A “nucleic acid” is a deoxyribonucleotide or ribonucleotide polymer in either single- or double-stranded form, and unless otherwise limited, encompasses known analogs of natural nucleotides that function in a manner similar to naturally occurring nucleotides.

An “oligonucleotide” is a nucleic acid polymer composed of two or more nucleotides or nucleotide analogues. An oligonucleotide can be derived from natural sources but is often synthesized chemically and can be of any size.

A “probe” is a nucleic acid sequence, e.g., a short shared nucleotide sequence (i.e., a differentiating nucleic acid probe) or a member of a sample nucleic acid population, that is hybridized, e.g., to an array of sample nucleic acids.

A “search and selection instruction set” is an algorithm used to search a nucleic acid population for nucleic acid sequences that can function as short shared nucleotide sequences. The search and selection parameters can be set by an operator. Once located, the short shared nucleotide sequences are, e.g., selected for subsequent use, e.g., in differentiating nucleic acid probe synthesis.

A “set” as used herein refers to a collection of at least two molecule or sequence types.

A “short shared nucleotide sequence” is a nucleic acid sequence (e.g., a restriction site, a homopolymer, a sequence repeat, or the like) that at least two members (e.g., expressed sequence tags, genes, or the like) of a population (e.g., a genome, a cDNA library, a DNA library, an RNA library, or the like) of nucleic acid sequences have in common. The short shared nucleotide sequence can be identical to, substantially identical to, complementary to, or substantially complementary to the corresponding sequence of the nucleic acid member. A “coding subsequence” corresponds to a subsequence that is represented in a mature RNA product. A “short shared polypeptide sequence” is a polypeptide sequence that at least two members of a polypeptide population have in common.

A “solid substrate” has a fixed organizational support matrix, such as nylon, nitrocellulose, silica, polymeric materials, glass, or the like. In some embodiments, at least one surface of the substrate is partially planar. In other embodiments it is desirable to physically separate regions of the substrate to delineate synthetic regions, for example with trenches, grooves, wells or the like. Examples of solid substrates include slides, beads and polymeric chips. A solid support is optionally “functionalized” to permit the coupling of monomers used in polymer synthesis. For example, a solid support is optionally coupled to a nucleoside monomer through a covalent linkage to the 3′-carbon on a furanose. Solid support materials typically are unreactive during polymer synthesis, providing a substrate to anchor the growing polymer. Solid support materials include, but are not limited to paper, nitrocellulose, nylon, glass, silica, controlled pore glass (CPG), polystyrene, polystyrene/latex, and carboxyl modified Teflon®. The solid substrates are biological, nonbiological, organic, inorganic, or a combination of any of these, existing as particles, strands, precipitates, gels, sheets, tubing, spheres, containers, capillaries, pads, slices, films, plates, slides, etc. depending upon the particular application. In light-directed synthetic techniques, the solid substrate is often planar but optionally takes on alternative surface configurations. For example, the solid substrate optionally contains raised or depressed regions on which synthesis takes place. In some embodiments, the solid substrate is chosen to provide appropriate light-absorbing characteristics. For example, the substrate may be a polymerized Langmuir Blodgett film, functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Other suitable solid substrate materials will be readily apparent to those of skill in the art. The surface of the solid substrate will optionally include reactive groups, such as carboxyl, amino, hydroxyl, thiol or the like. More preferably, the surface is optically transparent and has surface SiOH functionalities, such as are found on silica surfaces. A substrate is a material having a rigid or semi-rigid surface. In spotting or flowing techniques, at least one surface of the solid substrate is optionally planar, although in many embodiments it is desirable to physically separate synthesis regions for different polymers with, for example, wells, raised regions, etched trenches, or the like. In some embodiments, the substrate itself contains wells, trenches, flow through regions, etc. which form all or part of the regions upon which polymer synthesis occurs.

### BRIEF DESCRIPTION OF THE DRAWING

FIG. 1 is a schematic diagram showing how differentiating subsets of short shared nucleotide sequences can distinguish members of a nucleic acid population from one another.

FIG. 2 provides degenerate differentiating nucleic acid probe sets based on the BamHI restriction site.

### DETAILED DISCUSSION OF THE INVENTION

A major factor contributing to cDNA complexity stems from the divergence of primary nucleotide sequences in corresponding genes. Most genes include distinctive subsequences. However, many genes also share similar, if not identical, subsequences. Examples of shared sequences can be found among the members of certain multigene families, among unrelated genes that share common domains or motifs, or among completely unrelated genes that simply include various sequence motifs.

Restriction enzyme recognition sites are a simple example of short shared nucleotide sequences. The distribution and frequency of short shared nucleotide sequences can be identified and assessed through nucleic acid databases, e.g., EST databases. Specific sets of these known sequences can differentiate members, e.g., ESTs or genes, of nucleic acid populations from one another. In turn, synthesized sequences corresponding to sets of short shared nucleotide sequences can be utilized as probes to identify target sequences in samples containing unknown nucleotide sequences, to evaluate gene expression levels across various cell or tissue types, and to identify polymorphisms, among others applications.

In overview, the present invention provides methods of selecting sets of short shared nucleotide sequences from amongst the members of nucleic acid populations and of identifying subsets of those selected short shared nucleotide sequences that differentiate those members from one another. Probes corresponding to the sets of short shared nucleotide sequences can be synthesized and utilized, inter alia, to detect target nucleic acids in a sample population or to identify allelic variants of a gene. The invention also includes integrated systems for performing various steps involved in the methods and certain probe compositions.

The following provides details regarding the various aspects of the short shared nucleotide sequence selection methods of the present invention, including selection, synthesis, and arraying protocols. It also provides details pertaining to the integrated systems and different probe compositions of the present invention.

Short Shared Nucleotide Sequence Selection

Nucleic Acid Sequence Database Search

Many nucleic acid sequence databases, including various EST sequence databases, contain searchable sequence information that can be useful during the initial short shared nucleotide sequence selection process. Genbank®, Entrez®, EMBL, DDBJ, GSDB, NDB, and the NCBI are examples of public database/search services that can be accessed. Many sequence databases are available via the internet or on a contract basis from a variety of companies specializing in genomic information generation and/or storage. These and other helpful resources are readily available and known to those of skill.

Selection Criteria

Short Shared Nucleotide Sequence Types

The specific type of short shared nucleotide sequence included in a set of selected sequences depends primarily upon whether it ultimately contributes to the differentiation of members of the particular nucleic acid population under consideration from one another. Sequence-types that can potentially meet this criterion include restriction endonuclease recognition sequences (e.g., Alu I (AGCT), Alw44 I (GTGCAC), Apa I (GGGCCC), Asn I (ATTAAT), BamHI (GGATCC), Bcl I (TGATCA), Bgl II (AGATCT), Bln I (CCTAGG), Bss HII (GCGCGC), Cfo I (GCGC), Cla I (ATCGAT), Dde I (CTNAG), Dra I (TTTAAA), Ecl XI (CGGCCG), Eco RI (GAATTC), Eco RII (CC(A,T)GG), Eco RV (GATATC), Hae III (GGCC), Hind III (AAGCTT), Hinf I (GANTC), Hpa I (GTTAAC), Hpa II (CCGG), Kpn I (GGTACC), Ksp I (CCGCGG), Mlu I (ACGCGT), Mlu NI (TGGCCA), Msp I (CCGCr), Mva I (CC (A,T)GG), Nar I (GGCGCC), Nco I (CCATGG), Nde I (CATATG), Nhe I (GCTAGC), Not I (GCGGCCGC), Nru I (TCGCGA), Nsi I (ATGCAT), Pst I (CTGCAG), Pvu I (CGATCG), Pvu II (CAGCTG), Rsa I (GTAC), Sac I (GAGCTC), Sal I (GTCGAC), Sau 3A (GATC), Sca I (AGTACT), Scr FI (CCNGG), Sma I (CCCGGG), Spe I (ACTAGT), Sph I (GCATGC), Ssp I (AATATT), Stu I (AGGCCT), Swa I (ATTTAAAT), Taq I (TCGA), Xba I (TCTAGA), Xho I (CTCGAG), or the like), homopolymers (e.g., AAAAAA, CCCCCC, TTTTTT, GGGGGG, or the like), sequence repeats (e.g., ATATATAT, CTCCTCCTC, GCGCGCGC, or the like), and others.

Short regions of sequence identity, e.g., short shared nucleotide sequences, in a database can be determined by sequence alignment and comparison. In these processes of sequence alignment and comparison, one sequence is often used as a reference against which other test nucleic acid sequences are compared. This comparison can be accomplished with the aid of a sequence alignment and comparison or search and selection instruction set, i.e., algorithm, or by visual inspection. When an instruction set is employed, test and reference sequences are input into a computer, subsequence coordinates are designated, as necessary, and sequence algorithm program parameters are specified. The algorithm then calculates the percent sequence identity for the test nucleic acid sequence(s) relative to the reference sequence, based on the specified program parameters.

For purposes of the present invention, suitable sequence comparisons can be executed, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., supra).

One example of an algorithm that can be used to determine percent sequence identity and sequence similarity is the BLAST algorithm, which is described, e.g., in Altschul et al., (1990) J. Mol. Biol. 215: 403-410. Software for performing BLAST analyses is publicly available through the National Center for Biotechnology Information (http://www.ncbi.nlm.nih.gov/).

Although a short shared nucleotide sequence can be common to as few as two members of the nucleic acid population under consideration, this coverage density (i.e., the number of members to which a short shared nucleotide sequence is common) can be varied upwards, e.g., depending upon the particular nucleic acid population being investigated. The coverage density of a specific short shared nucleotide sequence can be, e.g., 5%, 10%, 15%, 20%, 25%, or more of all members of the nucleic acid population. The coverage density applicable in a particular case depends on the number of short shared nucleotide sequences in the set that is necessary to provide differentiating subsets of those sequences. The identification of differentiating subsets and the synthesis of differentiating nucleic acid probes based upon those subsets are discussed, infra.

There are various other identifiers from which short shared nucleotide sequences can be derived. In maize, for example, short shared nucleotid sequences can optionally be derived from consensus initiation sequences (i.e., MSSSSSMSSS SSMSRYMRCS ATGGCGRSSR YSR) or consensus termination sequences (i.e., SRMSRMSKMS TAARSRMYYM RWS).

Nucleic Acid Member Types

Short shared nucleotide sequences can be common to at least two members of a nucleic acid population. The members of nucleic acid populations can include various types of nucleic acid sequences based upon coding and/or non-coding regions of genes, and/or gene-associated elements. For example, members can include non-coding regions, such as introns, promoters, enhancers, or the like. Members can be coding regions that include segments of DNA involved in producing polypeptide chains and/or RNA chains, i.e., exons. Members can include regions preceding (e.g., leader) and following (e.g., trailer or terminal sequences) the coding region in addition to intervening non-coding sequences (e.g., introns) between individual coding segments. Additionally, members can include individual introns, exons, domains, motifs, expressed sequence tags, or the like. Nucleic acid population members can also include allelic variants of genes, e.g., polymorphisms. Furthermore, nucleic acid members can include operons, gene clusters, gene families, cloned nucleic acids (e.g., cosmids and BAC clones), or the like.

Nucleic Acid Population Types

The nucleic acid populations from which short shared nucleotide sequences can be selected include, e.g., a genome, a cDNA library, a DNA library, an RNA library, or the like. Furthermore, those populations can be derived from any organism (i.e., animals, plants, fungi, protists, and monera); in certain preferred embodiments they are derived from plants.

For example, a suitable nucleic acid population can be derived from essentially any plant. For example, nucleic acid populations can be derived from plants selected from the genera: Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Cichorium, Helianthus, Lactuca, Bromus, Asparagus, Antirrhinum, Heterocallis, Nemesia, Pelargonium, Panicum, Pennisetum, Ranunculus, Senecio, Salpiglossis, Cucumis, Browaalia, Lolium, Malus, Apium, Gossypium, Vicia, Lathyrus, Lupinus, Pachyrhizus, Wisteria, Stizolobium, or the like.

Important commercial crops include both monocots and dicots. Monocots include plants in the grass family plants (Gramineae), such as plants in the sub-families Fetucoideae and Poacoideae, which together include several hundred genera including plants in the genera Agrostis, Phleum, Dactylis, Sorghum, Setaria, Zea (e.g., corn), Oryza (e.g., rice), Triticum (e.g., wheat), Secale (e.g., rye), Avena (e.g., oats), Hordeum (e.g., barley), Saccharum, Poa, Festuca, Stenotaphrum, Cynodon, Coix, Olyreae, Phareae, Glycine, Pisum, Cicer, Phaseolus, Lens, Arachis, and many others. Additional commercially important crop plants are, e.g., from the families Compositae (the largest family of vascular plants, including at least 1,000 genera, including important commercial crops such as sunflower), and Leguminosae or “pea family,” which includes several hundred genera, including many commercially valuable crops such as pea, beans, lentil, peanut, yam bean, cowpeas, velvet beans, soybean, clover, alfalfa, lupine, vetch, sweet clover, wisteria, and sweetpea. Other common crops applicable to the method of the invention, include rapeseed and canola.

In the event nucleic acid sequence information, e.g., via a sequence database, is unavailable for the population of interest, the nucleic acid population can, e.g., be cloned into libraries and sequenced. These techniques are well-known and are discussed only briefly, infra.

Differentiating Subset Identification

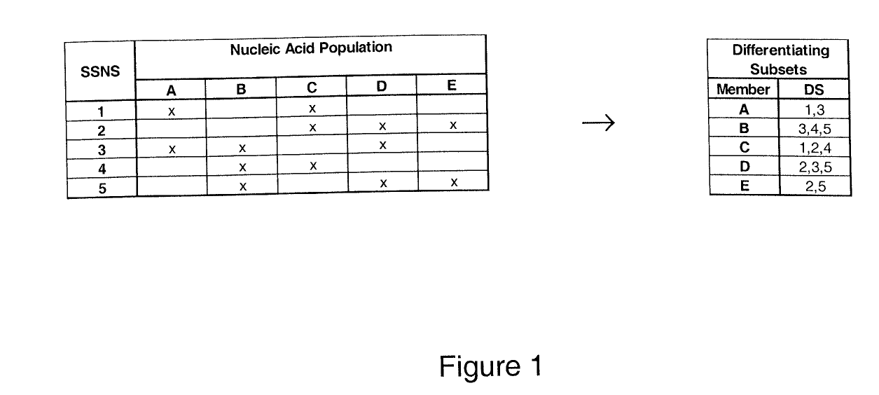

Once a set of short shared nucleotide sequences is selected from amongst the members of a nucleic acid population, differentiating subsets of those sequences can be identified. As shown in FIG. 1, differentiating subsets can be identified by searching for subsets of the selected set of short shared nucleotide sequences that are unique to individual members of the nucleic acid population. In a simple example, a nucleic acid population could include members, as follows;: A, B, C, D, and E. (FIG. 1). While the nucleic acid population could be, e.g., RNAs, DNAs and/or cDNAs, the members of that population could include one or more, e.g., expressed sequence tags, promoters, enhancers, exons, introns, domains, genes, polymorphisms, operons, gene clusters, gene families, and/or cloned nucleic acids.

By definition, each short shared nucleotide sequence is common to at least two members of the nucleic acid population and they can be selected as described, supra, with the aide of a sequence alignment and comparison instruction set, e.g., BLAST. Each short shared nucleotide sequence (SSNS) (i.e., in the simple example 17 depicted in FIG. 1: 1, 2, 3, 4, or 5) depicted meets this definitional requirement, as the symbol “x” indicates that the particular member has the corresponding short shared nucleotide sequence.

Once a set of short shared nucleotide sequences is selected, subsets of the set are identified that are unique to each individual member of the nucleic acid population. These subsets are differentiating subsets, because in the various methods of the present invention they can be utilized to distinguish members of nucleic acid populations from one another. For instance, upon detecting the hybridization of a set of probes that corresponds to a differentiating subset of short shared nucleotide sequences, the presence of a target member is indicated in a sample of a nucleic acid population. So, in FIG. 1, the differentiating subsets (DS) corresponding to each member are as follows: A (1,3), B (3,4,5), C (1,2,4), D (2,3,5), and E (2,5).

The foregoing is a simple example illustrating the principles involved in specifying differentiating subsets of short shared nucleotide sequences and the probes corresponding thereto. However, there are certain variable factors that can have an impact on differentiating subset identification. These can include the number of short shared nucleotide sequences in the set (e.g., 2, 3, 4, 5, 10, 20, 30, 40, 50, 100, 10³, 10⁴, 10⁵, 10⁶, or more sequences), the nature of an individual short shared nucleotide sequence (e.g., its length and related thereto, its coverage density or frequency of occurrence amongst members of a nucleic acid population), and the number (e.g., genome size, etc.) and nature (e.g., expressed sequence tags, promoters, exons, introns, domains, genes, operons, etc.) of the members of the particular nucleic acid population under investigation. As such, the identification of differentiating subsets can be case specific and the above mentioned factors, inter alia, should be taken into consideration.

### Target Detection

Array of Short Shared Nucleotide Sequences or Members of Sample Nucleic Acid Populations

In various embodiments of the methods of the present invention either a set of short shared nucleotide sequences or members of a sample nucleic acid population can be arrayed on a substrate surface to facilitate hybridization and detection. For a discussion of the various types of substrate surfaces applicable to the present invention, see “Substrates,” infra. Some arrays, e.g., “DNA chips” can include millions of defined nucleic acid regions on a substrate having an area of about 1 cm² to several cm². In addition to photomasking technologies, arrays of chemicals, nucleic acids, proteins or the like can also be printed on a solid substrate using printing technologies. The synthesis of nucleic acid arrays appropriate to the present invention is generally known. As such, no attempt is made to describe or catalogue all known methods. For exemplary purposes, light directed methods are briefly described, infra. One of skill will understand that alternate methods of creating nucleic acid arrays, such as spotting and/or flowing reagents over defined regions of a solid substrate, bead-based methods and pin-based methods are also known and applicable to the present invention. In the methods disclosed in these applications, reagents are typically delivered to the substrate by flowing or spotting polymer synthesis reagents on predefined regions of the solid substrate. See, e.g., Chee et al. (1996) Science 274: 610-614; Pease et al. (1994) Proc. Natl. Acad. Sci. USA 91: 5022-5026; Schena et al. (1995) Science 270: 467-470; and U.S. Pat. No. 5,807,522.

The light directed methods typically proceed by activating predefined regions of a substrate or solid support and then contacting the substrate with a preselected monomer solution. The predefined regions are activated with a light source, typically shown through a photolithographic mask. Other regions of the substrate remain inactive because they are blocked by the mask from illumination. Thus, a light pattern defines which regions of the substrate react with a given monomer. By repeatedly activating different sets of predefined regions and contacting different monomer solutions with the substrate, a diverse array of oligonucleotides is produced on the substrate. Other steps, such as washing unreacted monomer solution from the substrate, are used as necessary.

The surface of a solid support is typically modified with linking groups having photolabile protecting groups (e.g., NVOC or MeNPoc) and illuminated through a photolithographic mask, yielding reactive groups (e.g., typically hydroxyl groups) in the illuminated regions. For instance, during oligonucleotide synthesis, a 3′-O-phosphoramidite (or other nucleic acid synthesis reagent) activated deoxynucleoside (protected at the 5′-hydroxyl with a photolabile group) is presented to the surface and coupling occurs at sites that were exposed to light in the previous step. Following capping, and oxidation, the substrate is rinsed and the surface illuminated through a second mask, to expose additional hydroxyl groups for coupling. A second 5′-protected, 3′-O-phosphoramidite activated deoxynucleoside (or other oligonucleotide monomer as appropriate) is then presented to the resulting array. The selective photodeprotection and coupling cycles are repeated until the desired set of oligonucleotides is produced.

A number of other publications describe subject matter related to arraying technologies, e.g., Li, L. H. et al., (1999) Devel. Biol. 211: 64-76; Alon, U. et al., (1999) Proc. Natl. Acad. Sci. USA 96: 6745-6750; Fambrough, D. et al., (1999) Cell 97: 727-741; Zhu, H. et al., (1998) Proc. Natl. Acad. Sci. USA 95: 14470-14475; DeSaizieu, A. et al., (1998) Nat. Biotech. 16: 45-48; Cargill, M. et al., (1999) Nat. Genet. 22: 231-238; Halushka, M. et al., (1999) Nat. Genet. 22: 239-247; Hacia, J. G. et al., (1999) Nat. Genet. 22: 164-167, 1999; Sapolsky, R. et al., (1999) Genet. Anal.-Biomol. Engin. 14: 187-192;Gentalen, E., and Chee, M. (1999) Nucleic Acids Res. 27: 1485-1491; Wang, D. G. et al., (1998) Science 280: 1077-1082; Gingeras, T. R. et al., (1998) Genome Res. 8: 435-448; Hacia, J. G. et al., (1998) Nat. Genet. 18: 155-158; Fan, J. et al., (1997) Am. J. Human Gen. 61: 1601-1601; Ahrendt, S. A. et al., (1999) Proc. Natl. Acad. Sci. USA 96: 7382-7387; Alon, U. et al., (1999) Proc. Natl. Acad. Sci. USA 96: 6745-6750; Troesch, A. et al., (1999) J. Clin. Microbiol. 37: 49-55; Hacia, J. G. et al. (1998) Genome Res. 8: 1245-1258; Bulyk, M. L. et al., (1999) Nat. Biotech. 17: 573-577; Lipshutz, R. J. et al., (1999) Nat. Genet. (Microarray Supplement) 21: 20-24; and Hacia, J. G. et al., (1998) Nucleic Acids Res. 26: 4975-4982.

In addition to the large arrays described, supra, other arrays can also be made. For instance, standard Southern or northern blotting technology can be used to fix nucleic acid sequences to various substrates such as paper, nitrocellulose, nylon or the like. For example, as in the arraying formats discussed above, the members in a sample of an RNA or a DNA population can be spotted onto any of the mentioned substrates and differentiating nucleic acid probes hybridized, e.g., sequentially, thereto. These and other arraying techniques applicable to the present invention are described further in, e.g., Ausubel, supra. See also, Sambrook et al. (1989) Molecular Cloning—A Laboratory Manual (2nd ed.) Vol. 1-3, Cold Spring Harbor Laboratory, Cold Spring Harbor Press, NY, (Sambrook) and Berger and Kimmel, Guide to Molecular Cloning Techniques, Methods in Enzymology volume 152 Academic Press, Inc., San Diego, Calif. (Berger).

Preparing Short Shared Nucleotide Sequences and Members of Nucleic Acid Populations to be Coupled into Arrays; Synthesis of Nucleic Acids; Cloning of Nucleic Acids into Cells

As described, supra, several methods for the synthesis of nucleic acid arrays are know. In certain preferred embodiments, the oligonucleotides are synthesized directly on a solid surface as mentioned, supra. However, in certain embodiments, it is useful to synthesize the oligonucleotides and then to couple the oligonucleotides to the solid substrate to form the desired array. Similarly, nucleic acids in general (e.g., short shared nucleotide sequences for use as differentiating nucleic acid probes) can be synthesized on a solid substrate and then cleaved from the substrate, or they can be synthesized in solution (using chemical or enzymatic procedures), or they can be naturally occurring (i.e., present in a biological sample of a nucleic acid population).

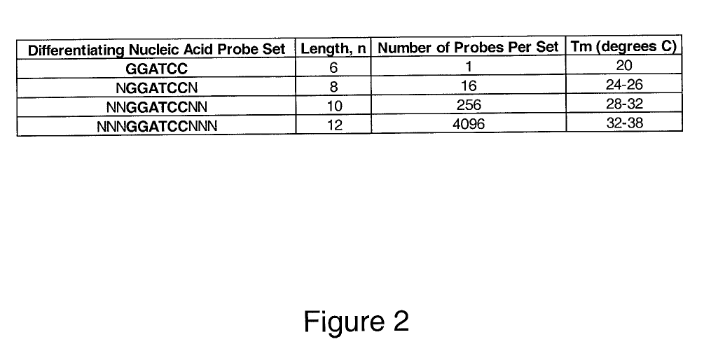

As shown in FIG. 2, differentiating nucleic acid probes can be synthesized with different levels of degeneration to satisfy the detection requirements of short shared nucleotide sequences in a significant portion of different genes. For example, a six base pair restriction site like BamHI (GGATCC) can be found on average once in every 4096 base pairs assuming GC/AT=1. (FIG. 2). If one further assumes that the average size of a full-sized cDNA, e.g., from the maize genome, is about 1000 bases in length, then a BamHI site will occur in every fourth EST. To identify those clones that include BamHI sites, degenerate sets of probes can be synthesized.

The specific activity and concentration of degenerate probe sets can be sufficient to detect corresponding sequences in a DNA sample even in the case of a degeneration level as high as 4096. (FIG. 2). Hybridization of such a set of probes to, e.g., an array of cDNA probes (e.g., purified plasmids or PCR products) can identify all clones that have at least one BamHI restriction site. Aside from restriction sites, degenerate probes can be synthesized based upon any nucleotide sequence, e.g., homopolymers and simple sequence repeats. Differentiating nucleic acid probes can further be designed in such a way so as to eliminate certain classes of sequences that are rare or non-informative, or have to be adapted to a specific melting temperature, Tₘ. (FIG. 2). Additionally, the GC/AT composition, the codon usage pattern, or certain organism-specific motifs, e.g., in the maize genome, can be helpful in designing appropriate sets of differentiating nucleic acid probes.

Molecular cloning and expression techniques for making biological and synthetic oligonucleotides and nucleic acids are known in the art. A wide variety of cloning and expression and in vitro amplification methods suitable for the construction of nucleic acids are well-known to persons of skill. Examples of techniques and instructions sufficient to direct persons of skill through many cloning exercises for the expression and purification of biological nucleic acids (DNA and RNA) are found in Ausubel, Sambrook and Berger, supra. Nucleic acids such as the members of sample nucleic acid populations can be cloned into cells (thereby creating recombinant cells) using standard cloning protocols such as those described in Ausubel, Sambrook and Berger, supra.

Examples of techniques sufficient to direct persons of skill through in vitro methods of nucleic acid synthesis and/or amplification of the members of a nucleic acid population and/or the set of differentiating nucleic acid probes in solution, including enzymatic methods such as the polymerase chain reaction (PCR.), the ligase chain reaction (LCR), Qβ-replicase amplification (QBR), nucleic acid sequence based amplification (NASBA), strand displacement amplification (SDA), the cycling probe amplification reaction (CPR), branched DNA (bDNA) and other DNA and RNA polymerase mediated techniques are known. Examples of these and related techniques are found in Ausubel, Sambrook, and Berger, as well as Mullis et al., (1937) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990); WO 94/11383; Vooijs et al., (1993) Am J. Hum. Genet. 52: 586-597; C&EN 36-47; The Journal Of NlH Research (1991) 3, 81-94; Kwoh et al., (1989) Proc. Natl. Acad. Sci. USA 86, 1173; Guatelli et al., (1990) Proc. Natl. Acad. Sci. USA 87, 1874; Lomell et al., (1989) J. Clin. Chem 35, 1826; Landegren et al., (1988) Science 241, 1077-1080; Van Brunt (1990) Biotechnology 8, 291-294; Wu and Wallace (1989) Gene 4, 560; Sooknanan and Malek (1995) Bio/Technology 13, 563-564; and Barringer et al., (1990) Gene 89, 117. Improved methods of cloning in vitro amplified nucleic acids are described in Wallace et al., U.S. Pat. No. 5,426,039. In one preferred embodiment, the members of the sample nucleic acid population are amplified prior to hybridization with the various arrays as described above. For instance, where the members of a nucleic acid population are cloned into cells in a cellular library, the members can be amplified using PCR.

Standard solid phase synthesis of nucleic acids is also known. This can be used, inter alia, to synthesize differentiating nucleic acid probes and/or competitor differentiating nucleic acid probes. Oligonucleotide synthesis is optionally performed on commercially available solid phase oligonucleotide synthesis machines (see, Needham-VanDevanter et al. (1984) Nucleic Acids Res. 12:6159-6168) or manually synthesized using the solid phase phosphoramidite triester method described by Beaucage et. al., (1981) Tetrahedron Letts. 22 (20): 1859-1862). Finally, as described supra, nucleic acids can be optionally synthesized using certain array-based methods, and optionally cleaved from the array. The nucleic acids can then be optionally reattached to a solid substrate to form a second array where appropriate, or used as differentiating nucleic acid probes where appropriate, or used as members of a nucleic acid population for cloning into a cell.

Furthermore, essentially any nucleic acid can be custom ordered from any of a variety of commercial sources, such as The Midland Certified Reagent Company (mcrc@oligos.com), The Great American Gene Company (http://www.genco.com), ExpressGen, Inc. (www.expressgen.com), Operon Technologies, Inc. (www.operon.com) and many others.

Substrates

As mentioned supra, depending upon the assay, the members of a sample nucleic acid population, or the short shared nucleotide sequences can be bound to a solid surface. Many methods for immobilizing nucleic acids to a variety of solid surfaces are known in the art. For instance, the solid surface is optionally paper, or a membrane (e.g., nitrocellulose), a microtiter dish (e.g., PVC, polypropylene, or polystyrene), a test tube (glass or plastic), a dipstick (e.g. glass, PVC, polypropylene, polystyrene, latex, and the like), a microcentrifuge tube, or a glass, silica, plastic, metallic or polymer bead or other substrate as described herein. The desired component may be covalently bound, or noncovalently attached to the substrate through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, or the like. Other materials which are appropriate depending on the assay include ceramics, metals, metalloids, semiconductive materials, cements or the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts or the like are also suitable. Where the solid surface is porous, various pore sizes may be employed depending upon the nature of the system.

In preparing the surface, a plurality of different materials are optionally employed, e.g., as laminates, to obtain various properties. For example, protein coatings, such as gelatin can be used to avoid non-specific binding, simplify covalent conjugation, enhance signal detection or the like. If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups, or the like. In addition to covalent bonding, various methods for noncovalently binding an assay component can be used.

Labeling, Hybridization, and Detection

In various methods of the invention, as described supra, micleic acid sequences (e.g., a set of short shared nucleotide sequences or members of a nucleic acid population) can be, e.g., chemically linked in an array to a solid support and nucleic acid probes (e.g., a set of differentiating nucleic acid probes) can be hybridized to the array. As a first embodiment of these methods, the nucleic acid probes can be hybridized sequentially, i.e., one at a time, to the arrayed nucleic acid sequences and the hybridization patterns detected at each sequential hybridization. Either the array, or the probes, or both, can be labeled, typically with a fluorophore. Where the probes are labeled, hybridization can be detected by detecting bound fluorescence. Where the probes are labeled, hybridization can be detected by quenching the label by the bound nucleic acid. Where both the probe and the target are labeled, detection of hybridization can be performed by monitoring a signal shift such as a change in color, fluorescent quenching, or the like, resulting from proximity of the two bound labels.

A wide variety of labels suitable for labeling nucleic acids and conjugation techniques are known and are reported extensively in both the scientific and patent literature, and are generally applicable to the present invention for the labeling of nucleic acids for detection of hybridization to the arrays of the invention. Suitable labels include radionucleotides, enzymes, substrates, cofactors, inhibitors, fluorescent moieties, chemiluminescent moieties, magnetic particles, or the like. Labeling agents optionally include, e.g., monoclonal antibodies, polyclonal antibodies, proteins, or other polymers such as affinity matrices, carbohydrates or lipids. Detection of nucleic acid probes proceeds by any known method, including immunoblotting, tracking of radioactive or bioluminescent markers, Southern blotting, northern blotting, southwestern blotting, northwestern blotting, or other methods which track a molecule based upon size, charge or affinity. The particular label or detectable group used and the particular assay are not critical aspects of the invention. The detectable moiety can be any material having a detectable physical or chemical property. Such detectable labels have been well-developed in the field of gels, columns, solid substrates and in general, labels useful in such methods can be applied to the present invention. Thus, a label is any composition detectable by spectroscopic, photochemical, biochemical, immunochemical, electrical, optical or chemical means. Useful labels in the present invention include fluorescent dyes (e.g., fluorescein isothiocyanate, Texas red, rhodamine, and the like), radiolabels (e.g., ³H, ³⁵S, ¹⁴C, ³²P, or the like), calorimetric labels, or the like.

The label is coupled directly or indirectly to the desired nucleic acid according to methods well known in the art. As indicated above, a wide variety of labels are used, with the choice of label depending on the sensitivity required, ease of conjugation of the compound, stability requirements, available instrumentation, and disposal provisions. Non-radioactive labels are often attached by indirect means. Generally, a ligand molecule (e.g., biotin) is covalently bound to a polyrrer. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers, or the like. For detection in arrays, for example, fluorescent labels and detection techniques, particularly microscopy are preferred. Similarly, enzymatic labels may be detected by providing appropriate substrates for the enzyme and detecting the resulting reaction product. Finally, simple calorimetric labels are often detected simply by observing the color associated with the label. Thus, in various dipstick assays, conjugated gold often appears pink, while various conjugated beads appear the color of the bead.

Non-array-based Detection

There are various alternatives to the array-based sequential hybridization embodiment mentioned above, including high-throughput microfluidics devices and gel-based formats, that can be used where one label at a time is detected. For example, the HP 2100 Bioanalyzer Nucleic Acid Analysis System is an integrated system that incorporates microfluidic devices developed by Caliper Technologies Corp. (www.calipertech.com) which can be used for sequential hybridizations. As applied to the present invention, this technique involves flowing, e.g., each member of a nucleic acid population exposed sequentially to each labeled differentiating nucleic acid probe in a set, through a channel on a LabChip™ and detecting whether hybridization occurs. Based upon the hybridization data gathered during this process, once a particular member of the population has been exposed to all the labeled probes, it can potentially be identified according the set of probes to which it hybridized.

In a gel-based format, nucleic acid population members can be electrophoresed through, e.g., an agarose gel, and exposed to one labeled differentiating nucleic acid probe at a time and any hybridization can be detected. Thereafter, the set of probes that hybridized to each position on the gel can be used to identify the members of the nucleic acid population at those specific positions.

Applications of Differentiating Subsets of Short Shared Nucleotide Sequences

The methods of the present invention have many applications, including gene identification, expression profiling, or the like which will be recognized by those of skill. As such, no attempt is made to describe or catalogue all known uses. However, for exemplary purposes, some applications of the present invention are briefly described below.

Parallel Gene Identification in a cDNA Library

Oligonucleotide probes, e.g., differentiating nucleic acid probes, can be synthesized and hybridized consecutively to an arrayed cDNA library (e.g., purified plasmid DNA or amplified inserts). Each probe can have a certain number of hits (e.g., 5%, 10%, 15%, 20%, 25% or more) in such arrayed libraries. From the sets of differentiating nucleic acid probes that hybridize to the arrayed cDNA library, one can identify specific genes present in the library. Probes can also be labeled with different fluorochromes to derive multiplex advantages. See, generally the above discussion with respect to, e.g., different arraying formats, probe synthesis techniques, and the various cited references. See also, the sections related to expression profiling, infra.

Individual Gene Identification

One variation of the method of parallel gene identification involves arraying a set of probes that correspond to a set of short shared nucleotid sequences (e.g., restriction enzymes sites, homopolymers, sequence repeats, and the like). In this embodiment, a cDNA can then be hybridized to the arrayed set of short shared nucleotide sequences. One can identify genes using this format based upon the specific short shared nucleotide sequence probes to which a particular cDNA hybridizes in the array.

Competitor DNA in Hybridization Experiments

Non-specific cross-hybridization can be a significant problem in many hybridization experiments. Oligonucleotide probes that are complementary to a set of short shared nucleotide sequences (e.g., competitor differentiating nucleic acid probes) can be used to minimize, if not eliminate, non-specific cross-hybridization. For example, in Southern or northern hybridization experiments, competitor differentiating nucleic acid probes can be concomitantly hybridized with a set of differentiating nucleic acid probes to the arrayed target nucleic acids. Differentiating nucleic acid probes (or DNAPs) that do not hybridize to arrayed target nucleic acids can hybridize instead to competitor differentiating nucleic acid probes (or CDNAPs) to form DNAP-CDNAP hybrid molecules that can simply be washed from the array to prevent non-specific cross-hybridization. Competitor differentating nucleic acid probes can also be used in this manner to at least minimize non-specific cross-hybridization in hybridization experiments conducted on microchips or in any other array format.

Gene (EST) Assignment on Cloned Genomic DNA Fragments

The methods of the present invention can also be used to identify genes in cloned genomic DNA fragments. For example, oligonucleotide probes, such as, differentiating nucleic acid probes, can be synthesized and hybridized consecutively to an arrayed genomic DNA library (e.g., cosmids or BACs). Each differentiating nucleic acid probe can have a certain number of hits (e.g., 5%, 10%, 15%, 20%, 25% or more) in such an arrayed library. From the sets of differentiating nucleic acid probes that hybridize to the arrayed library, one can identify specific genes present in the library.

CDNA Library Normalization

Differentiating nucleic acid probes can also be used to identify clones with identical short shared nucleotide sequence signatures, i.e., which hybridize to the same set of probes and in turn, those clones can be reduced to one representative member. For example, in this embodiment of the invention, a target nucleic acid sequence (e.g., a cDNA) can be detected at least twice by identifying members of a nucleic acid population that hybridize to the same set of differentiating nucleic acid probes. This can help, inter alia, to minimize resequencing when directly prepared cDNA libraries are used.

Detection of DNA Polymorphisms in Parallel Mode Across all ESTs

At a certain level of short shared nucleotide sequence density, i.e., coverage density, the probability of unambiguous identification of a specific gene can be high enough to enable one to further identify allelic variants. For example, if an EST has 20 short shared nucleotide sequences, of which only five are sufficient for purposes of gene identification, the remaining 15 can then be used to identify mutations. Thus, a polymorphism can potentially be identified by detectirg the hybridization of at least one differentiating nucleic acid probe (i.e., at least one of the remaining 15) to an individual member of a nucleic acid population in addition to the hybridization of the differentiating subset of probes that identifies the individual member. This is only a simple example for purposes of illustration, in practice the detection of polymorphisms can require 100 or more short shared nucleotide sequences.

Mapping

A nucleic acid population can correspond to a multigene family and detected polymorphisms (discussed above) can be used to map a member within a nucleic acid population by linkage analysis. For general information about linkage analysis see, Watson et al., Recombinant DNA, ₂nd Ed., W.H. Freeman and Company, New York (1992) and Lewin, Genes VI, Oxford University Press, Inc., New York (1997).

Comparative Analysis of Gene Expression Levels for Alleles and Members of Multigene Families

At a certain coverage density (i.e., the number of members of a nucleic acid population to which a short shared nucleotide sequence is common), at least some alleles of a gene or members of a multigene family can be identified utilizing one or more of the various embodiments of the present invention discussed above. The efficiency of expression of an allelic variant of a particular gene can then be determined. The expression levels of the various members of the multigene family can also be determined. For further discussion of expression analysis, see Expression Profiling, infra.

Compatibility of Short Shared Nucleotide Sequence Profiling with LYNX Technology

In another embodiment of the present invention, fluorochrome labeled differentiating nucleic acid probes can be hybridized to DNA sequences linked to beads. Furthermore, multiplexing can also be accomplished in this embodiment. As described above, consecutive rounds of hybridization of different differentiating nucleic acid probes to linked DNA sequences can lead to gene identification. Following identification, different classes of DNA linked beads can be fractionated based upon their short shared nucleotide sequence profiles.

Use of Short Shared Nucleotide Sequences in Cosmid and BAC Clone Profiling

The hybridization methods described above can also be applied to the analysis of cosmid and BAC clones, many of which include similar or identical repetitive elements that can be used in clone identification. As these clones typically include multiple 6-mer restriction sites (e.g., 25 sites per 100 kb), 8-mer and even 10-mer short shared nucleotide sequences can be used as they occur in 1-4 clones on average. Differentiating nucleic acid probes can be synthesized as described above (e.g., NNNGCGGCCGCNNN, Tm 36-48° C.). This will enable one to determine the distribution of less frequently occurring restriction sites in parallel in all clones in a library (e.g., purified plasmid DNA samples).

Expression Profiling

A cDNA library (e.g., an arrayed cDNA library) can be analyzed with a set of short shared nucleotide sequences (e.g., a set of differentiating nucleic acid probes) to reveal what types of genes have been expressed. If such a library is non-normalized, this method can enable one to estimate the level of expression of different genes. Comparison among cDNA libraries derived from RNA isolated from different tissues or after being subjected to various experimental conditions can also be done using this method, as discussed further below.

A variety of tissues can be profiled, but much of the discussion herein relates to commercially valuable crops, as these are an important target of the method of the invention. However, the method is general and can be applied to non-commercial crop plants, fungi, protists, monera, and to the production of animals, including poultry, cattle, sheep, pigs, and the like. See also, Nucleic Acid Population Types, supra. As for plants, immature tissues are preferred, because it increases the rate at which crops can be screened, as a plant does not have to be grown to maturity. Nonetheless, essentially any tissue, or whole plant, can be profiled. A variety of profiling methods are available, including hybridization of expressed or amplified nucleic acids to a nucleic acid array, hybridization of expressed polypeptides to a protein array, hybridization of peptides or nucleic acids to an antibody array, subtractive hybridization, differential display and others.

RNA Profiling

In one preferred embodiment, the expression products which are detected by the methods of the invention are RNAs, e.g., mRNAs expressed from genes within a cell of the plant or tissue to be profiled. A number of techniques are available for detecting RNAs. For example, northern blot hybridization is widely used for RNA detection, and is generally taught in a variety of standard texts on molecular biology, including: Berger, Sambrook, and Ausubel, supra. Furthermore, one of still will appreciate that essentially any RNA can be converted into a double stranded DNA suitable for restriction digestion, PCR expansion and sequencing using a reverse transcriptase enzyme and a polymerase. See, Berger, Sambrook and Ausubel, supra. Thus, detection of mRNAs can be performed by converting, e.g., mRNAs into DNAs, which are subsequently detected in, e.g., a standard “Southern blot” format.

As mentioned supra, DNAs can also be amplified to aid in the detection of rare molecules by any of a number of well known techniques, including: the polymerase chain reaction (PCR), the ligase chain reaction (LCR), Qβ-replicase amplification and other RNA polymerase mediated techniques (e.g., NASBA).

These general methods can be used for expression profiling. For example, arrays of nucleic acid sequences, e.g., expression products (or in vitro amplified nucleic acids corresponding to expression products), can be arrayed on a surface and short shared nucleotide sequences can be labeled and hybridized with the array. For convenience, it may be helpful to use several arrays simultaneously. It is expected that one of skill is familiar with nucleic acid hybridization. General methods of hybridization are found in Berger, Sambrook, and Ausubel, supra, and, further in Tijssen, supra. Furthermore, the expression products can be compared between two cell populations as one way to identify mRNA species which are differentially expressed between the cell populations (i.e., present at different abundances between the cell populations).

Protein Profiling

It is contemplated that differentiating subsets of short shared polypeptide sequence sets can be used to distinguish members, e.g., proteins, of a polypeptide population from one another. As such, in addition to profiling RNAs (or corresponding cDNAs) as described supra, it is also possible to profile proteins.

Similar to selecting sets of short shared nucleotide sequences, sets of short shared polypeptide sequences can be selected from amongst members of a polypeptide population. The short shared polypeptide sequences can include amino acid subsequences that are common to at least two members of the polypeptide population. Thereafter, differentiating subsets of the set of short shared polypeptide sequences can be identified to distinguish members of the polypeptide population from one another. The steps of short shared polypeptide sequence selection and differentiating subset identification can, e.g., be performed in silico. Furthermore, identified differentiating subsets can then be used, e.g., to determine the presence of a target polypeptide in a sample population of unknown polypeptides. For example, native proteins can be fragmented (e.g., using a protease) or denatured (e. ., using urea or guanidinium HCl) to expose amino acid sequences corresponding to short shared polypeptide sequences which can function, e.g., as epitopes for antibody recognition. Antibodies, e.g., in an array or otherwise can then be used to detect differentiating subsets of short shared polypeptide sequences and hence, the presence of a target protein.

As applied to the present invention, detected proteins, corresponding to expression products, can be derived from one of at least two sources. First, the proteins which are detected can be either directly isolated from a cell or tissue to be profiled, providing direct detection (and, optionally, quantification) of proteins present in a cell. Second, mRNAs can be translated into cDNA sequences, cloned and expressed. This increases the ability to detect rare RNAs, and makes it possible to immediately associate a detected protein with its coding sequence.

A variety of hybridization techniques, including western blotting, ELISA assays, and the like are available for detection of specific proteins. See, Berger, Sambrook, and Ausubel, supra. See also, Antibodies: A Laboratory Manual, (1988) E. Harlow and D. Lane, Cold Spring Harbor Laboratory, Cold Spring Harbor, N.Y. Non-hybridization based techniques such as two-dimensional electrophoresis can also be used to simultaneously and specifically detect large numbers of proteins.

One typical technology for detecting specific proteins involves making antibodies to the proteins, e.g., to short shared polypeptide sequences. By specifically detecting binding of an antibody and a given protein, the presence of the protein can be detected. In addition to available antibodies, one of skill can easily make antibodies using existing techniques, or modify those antibodies which are commercially or publicly available. In addition to the art referenced above, general methods of producing polyclonal and monoclonal antibodies are known to those of skill in the art. See e.g., Paul (ed) (1993) Fundamental Immunology, Third Edition Raven Press, Ltd., New York Coligan (1991) Current Protocols in Immunology Wiley/Greene, NY; Harlow and Lane (1989) Antibodies: A Laboratory Manual Cold Spring Harbor Press, NY; Stites et al., (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497. Other suitable techniques for antibody preparation include selection of libraries of recombinant antibodies in phage or similar vectors. See, Huse et al., (1989) Science 246: 1275-1281 and Ward et al., (1989) Nature 341: 544-546.

In one embodiment of the present invention, it is contemplated that antibodies or antibody fragments can be arrayed, e.g., by coupling to an amine moiety fixed to a solid phase array, in a manner similar to that described supra for construction of nucleic acid arrays. As for arrayed nucleic acid sequences, antibodies can be labeled, or proteins corresponding to expression products can be labeled. In this manner, it is possible to couple hundreds, or even thousands, of different antibodies to an array.

The patterns of hybridization which are detected provide an indication of the presence or absence of protein sequences. As long as the library or array against which a population of proteins are to be screened can be correlated from one experiment to the next (e.g., by noting the x-y coordinates of the library or array member), no sequence information is required to compare expression profiles from one representative sample to another. In particular, the mere presence or absence (or degree) of label provides the ability to determine differences. One advantage of using libraries of antibodies for protein detection is that the individual libraries can be uncharacterized. As long as library members have a set spatial relationship, e.g., gridded on a plate, duplicate plates can be made and label patterns to the set spatial relationship determined.

More generally, peptide and nucleic acid hybridization to arrays or libraries (or even simple two dimensional gels) can be treated in a manner analogous to a bar code label. Any diverse library or array can be used to screen for the presence or absence of complementary molecules, whether RNA, DNA, protein, or a combination thereof. By measuring corresponding signal information between different sources of test material (e.g., different hybrid or inbred plants, or different tissues, or the like), it is possible to determine differences in expression products for the different source materials. As set forth infra, this process is facilitated by various high throughput integrated systems.

In addition to array based approaches, mass spectrometry is in use for identification of large sets of proteins in samples, and is suitable for identification of many proteins in a sequential or parallel fashion. It is contemplated that this technology can be utilized not only with respect to short shared polypeptide sequences, but also with respect to short shared nucleotide sequences. For example, Hutchens et al., U.S. Pat. 5,719,060, describe methods and apparatus for desorption and ionization of analytes for subsequent analysis by mass spectroscopy and/or biosensors.

Two and three dimensional gel based approaches can also be used for the specific and simultaneous identification and quantification of large numbers of proteins from biological samples. Multi-dimensional gel technology is well-known and described e.g., in Ausubel, supra, Volume 2, Chapter 10. Image analysis of multi-dimensional protein separation gels provides an indication of the proteins that are expressed, e.g., in a cell or tissue type.

Integrated Systems

The present invention includes an integrated system that includes a computer or computer readable medium that includes a database with at least one sequence record that includes one or more character strings corresponding to at least one nucleic acid or polypeptide population and to at least one set of short shared nucleotide or polypeptide sequences. The system also includes a user input interface that allows the user to selectively view the sequence record. Additionally, the system can include a sequence search and selection instruction set that searches the character strings corresponding to the members of the nucleic acid population and selects desired short shared nucleotide sequences from amongst the members. Among other things, the methods of gene identification discussed in this disclosure can be performed in silico using these integrated systems.

As discussed above, short shared nucleotide sequences can be determined by aligning and comparing sequences in a database. This method can also be applied to the selection of short shared polypeptide sequences. In this process of searching for nucleic acid or polypeptide sequences in common to two or more members of a population, one sequence is often used as a reference against which other test nucleic acid or polypeptide sequences are compared. This search and selection process can be accomplished in the integrated systems of the present invention with a sequence alignment and comparison or search and selection instruction set, i.e., algorithm. When such an instruction set is employed, test and reference sequences are input into a computer, subsequence coordinates are designated, as necessary, and sequence algorithm program parameters are specified. The algorithm then calculates the percent sequence identity for the test nucleic acid or polypeptide sequence(s) relative to the applicable reference sequence, based on the specified program parameters.

For purposes of the integrated systems present invention, as mentioned above, suitable sequence comparisons can be executed, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.) (see generally, Ausubel et al., supra).

One example of an algorithm that can be used to detect ungapped subsequences that match a given query sequence is the BLAST algorithm, which is described, e.g., in Altschul et al., (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, e.g., short shared nucleotide sequences, the parameters M (reward score for a pair of matching residues; always >0) and N (penalty score for mismatching residues; always <0). For amino acid sequences, e.g., when searching for short shared polypeptide sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). An operator can alter the algorithm's default parameters.

In one aspect, the invention provides an integrated system comprising a computer or computer readable medium comprising a database with at least one sequence record that includes one or more character strings corresponding to at least one nucleic acid or polypeptide population and to at least one set of short shared nucleotide or polypeptide sequences. The system also includes a user interface allowing a user to selectively view one or more sequence database programs for aligning and manipulating sequences. In addition, standard text manipulation software such as word processing software (e.g., Microsoft Word™ or Corel Wordperfect™) and database software (e.g., spreadsheet software such as Microsoft Excel™, Corel Quattro PrO™, or database programs such as Microsoft Access™ or Paradox™) can be used in conjunction with a user interface (e.g., a GUI in a standard operating system such as a Windows, Macintosh or Linux system) to manipulate strings of characters. As noted, specialized alignment software such as BLAST can also be included.

The integrated system of the invention can also include an automated synthesizer coupled to an output of the computer or computer readable medium. The automatic synthesizer can accept instructions from the computer or computer readable medium and those instructions can direct the synthesis of, e.g., sets of differentiating nucleic acid probes. As discussed further infra, the system can also include one or more robotic control elements for manipulating a set of differentiating nucleic acid probes or the members of a nucleic acid population.

In another embodiment of the invention, the computer or computer readable medium of the integrated system can include an instruction set for reverse-transcribing RNA sequences, selected from members of the nucleic acid population, into cDNA sequences. This embodiment can further include a search and selection instruction set that selects short shared nucleotide sequences from amongst the members of the nucleic acid population. The integrated system of the present invention can also include a user readable output element that displays desired short shared nucleotide sequences produced by the search and selection instruction set.

The invention also provides integrated systems for, inter alia, sample manipulation. A robotic liquid control armature for transferring solutions (e.g., plant cell extracts) from a source to a destination, e.g., from a microtiter plate to an array substrate, is optionally operably linked to the digital computer. An input device for entering data to the digital computer to control high throughput liquid transfer by the robotic liquid control armature and, optionally, to control transfer by the armature to the solid support is commonly a feature of the integrated system.

Integrated systems for hybridization analysis of the present invention typically include a digital computer with high-throughput liquid control software, image analysis software, data interpretation software, a robotic liquid control armature for transferring solutions from a source to a destination operably linked to the digital computer, an input device (e.g., a computer keyboard) for entering data to the digital computer to control high throughput liquid transfer by the robotic liquid control armature and, optionally, an image scanner for digitizing label signals from labeled probes hybridized, e.g., to expression products on a solid support operably linked to the digital computer. The image scanner interfaces with the image analysis software to provide a measurement of, e.g., differentiating nucleic acid probe label intensity upon hybridization to an arrayed sample nucleic acid population, where the probe label intensity measurement is interpreted by the data interpretation software to show whether, and to what degree, the labeled probe hybridizes to a label.

A number of well known robotic systems have also been developed for solution phase chemistries which can be used in the present invention, e.g., in differentiating nucleic acid probe synthesis or in certain arraying formats or in sample manipulation (e.g., where a nucleic acid sample is derived from a plant cell). These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass., Orca, Hewlett-Packard, Palo Alto, Calif., or the like) which mimic the manual synthetic operations performed by a scientist. Any of the above devices are suitable for use with the present invention. The nature and implementation of modifications to these devices (if any) so that they can operate as discussed herein with reference to the integrated system will be apparent to persons skilled in the relevant art.

Optical images, e.g., hybridization patterns viewed (and, optionally, recorded) by a camera or other recording device (e.g., a photodiode and data storage device) are optionally further processed in any of the embodiments herein, e.g., by digitizing the image and/or storing and analyzing the image on a computer. A variety of commercially available peripheral equipment and software is available for digitizing, storing and analyzing a digitized video or digitized optical image, e.g., using PC (Intel x86 or pentium chip-compatible DOS™, OS2™ WINDOWS™, WINDOWS NT™ or WINDOWS95™ based machines), MACINTOSH™, or UNIX based (e.g., SUN™ work station) computers.

One conventional system carries light from the specimen field, e.g., fluorescence emitted from probes hybridized to an array, to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

The method of the invention can also include inputting an expression profile for the analyzed organisms (e.g., sets of short shared nucleotide sequence hybridization information) into a database of expression profiles. This can be performed manually, but is more typically performed in an automated system. Computer databases of expression profile information can be quite large, with from a few up to several thousand profiles in the database.

An assay, kit or system utilizing a use of any one of the selection strategies, materials, components, methods or substrates hereinbefore described. Kits will optionally additionally comprise instructions for performing methods or assays, packaging materials, one or more containers which contain assay, device or system components, or the like.

In an additional aspect, the present invention provides kits embodying the methods and apparatus herein. Kits of the invention optionally comprise one or more of the following: (1) an arraying/detection device as described herein; (2) instructions for practicing the method described herein, and/or for operating, e.g., the short shared nucleotide sequence selection, the differentiating subset identification, the differentiating nucleic acid probe synthesis, and the array synthesis procedures herein; (3) one or more assay component(s); (4) a container for holding nucleic acids or enzymes, other nucleic acids, transgenic plants, animals, cells, or the like, and (5) packaging materials.

In a further aspect, the present invention provides for the use of any component or kit herein, for the practice of any method or assay herein, and/or for the use of any apparatus or kit to practice any assay or method herein.

Downstream Processing

Cloning of Expression Product Sequences into Bacterial Hosts

Any nucleic acid encoding an expression product identified as being of interest by the expression profiling techniques noted herein can be cloned. The various cloning methods are well-known. See, e.g., Ausubel, Sambrook, and Berger, supra. A plethora of kits are commercially available for the purification of plasmid; from bacteria. For their proper use, follow the manufacturer's instructions (see, for example, EasyPrep™, FlexiPrep™, both from Pharmacia Biotech; StrataClean™, from Stratagene; and, QIAexpress Expression SysteM™ from Qiagen). Additional basic procedures for sequencing, cloning and other aspects of molecular biology and underlying theoretical considerations are also found in Watson et al. (1992) Recombinant DNA, 2nd Edition, Scientific American Books, NY.

Transfecting and Manipulating Plant Cells

Methods of transducing plant cells with nucleic acids are generally available. In addition to Ausubel, Sambrook and Berger, useful general references for plant cell cloning, culture and regeneration include Payne et al., (1992) Plant Cell and Tissue Culture in Liquid Systems, John Wiley & Sons, Inc. New York, N.Y. (Payne); and Gamborg and Phillips (eds) (1995) Plant Cell, Tissue and Organ Culture, Fundamental Methods, Springer Lab Manual, Springer-Verlag (Berlin Heidelberg N.Y.) (Gamborg). A variety of Cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1999) from Sigma-Aldrich, Inc (St Louis, Mo.) (Sigma-LSRCCC) and, e.g., the Plant Culture Catalogue and supplement (1999) also from Sigma-Aldrich, Inc (St Louis, Mo.) (Sigma-PCCS).

The nucleic acid constructs of the invention can be introduced into plant cells, either in culture or in the organs of a plant by a variety of conventional techniques. For example, the DNA construct can be introduced directly into the genomic DNA of the plant cell using techniques such as electroporation and microinjection of plant cell protoplasts, or the DNA constructs can be introduced directly into plant cells using ballistic methods, such as DNA particle bombardment. Alternatively, the DNA constructs are combined with suitable T-DNA flanking regions and introduced into a conventional Agrobacterium tumefaciens host vector. The virulence functions of the Agrobacterium tumefaciens host directs the insertion of the construct and adjacent marker into the plant cell DNA when the cell is infected by the bacteria.

Regeneration of Transgenic Plants

Transformed plant cells which are derived by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, Macmillian Publishing Company, New York, (1983); and Binding, Regeneration of Plants, Plant Protoplasts, pp. 21-73, CRC Press, Boca Raton, (1985). Regeneration can also be obtained from plant callus, explants, somatic embryos (Dandekar et al., (1989) J. Tissue Cult. Meth. 12: 145; McGranahan et al., (1990) Plant Cell Rep. 8: 512), organs, or parts thereof. Such regeneration techniques are described generally in Klee et al., (1987) Ann. Rev. of Plant Phys. 38: 467-486.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Compositions

The present invention provides a method of selecting sets of short shared nucleotide sequences from amongst members of nucleic acid populations. Subsets of the selected short shared nucleotide sequences are then identified that differentiate members of the population from one another. Thereafter, probes corresponding to the identified differentiating subsets of the selected short shared nucleotide sequences can be synthesized as described, supra. These nucleic acid probes can then be employed in various applications. For example, labeled probes can be utilized to detect the presence of target nucleic acids in samples of unknown nucleic acid populations, e.g., an arrayed sample population, to assess gene expression across various tissue-types, to identify allelic variants of a gene, and the like. Further, competitor probes which are complementary to a set of differentiating nucleic acid probes can be synthesized and used, e.g., to minimize non-specific cross-hybridization of those sets of differentiating nucleic acid probes.

As such, the present invention also provides a composition that includes one or more libraries of differentiating nucleic acid probes that correspond to the set or sets of selected short shared nucleotide sequences. The short shared nuclcotide sequence selection and differentiating subset identification processes were described, supra. The libraries collectively include a plurality of differentiating nucleic acid probe member types. Differentiating subsets of the plurality of probe member types are unique to individual members of a nucleic acid population.

The invention further includes a composition that includes one or more libraries of competitor differentiating nucleic acid probes. As mentioned above, they correspond to a set of nucleic acid sequences that are complementary to a set of short shared nucleotide sequences, e.g., a set of differentiating nucleic acid probes.

Both the differentiating nucleic acid probe composition and the competitor differentiating nucleic acid probe composition can be cloned. As mentioned above, the assorted cloning techniques are well-known. See, e.g., Ausubel, Sambrook, and Berger, supra. A wide variety of cloning kits and associated products are commercially available from, e.g., Pharmacia Biotech, Stratagene, Sigma-Aldrich Co., Novagen, Inc., Fermentas, and 5 Prime→3 Prime, Inc.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of identifying differentiating subsets of short shared nucleotide sequences or of differentiating at least one target nucleic acid sequence from other members of a first nucleic acid population, the method comprising:
selecting a set of short shared nucleotide sequences from the members of the first nucleic acid population, wherein each short shared nucleotide sequence comprises a nucleic acid subsequence that is common to at least two members of the first nucleic acid population; and,
identifying differentiating subsets of the set of short shared nucleotide sequences, wherein each differentiating subset comprises a subset of the set of short shared nucleotide sequences which differentiates an individual member of the first nucleic acid population from the other members of the first nucleic population; or,
detecting one or more differentiating subsets of the set of short shared nucleotide sequences that correspond to the at least one target nucleic acid sequence, thereby differentiating the at least one target nucleic acid sequence from the other members of the first nucleic acid population.

2. The method of claim 1, wherein the selecting step comprises selecting the set of short shared nucleotide sequences from the members of the first nucleic acid population, wherein each short shared nucleotide sequence comprises a nucleic acid coding subsequence that is common to at least two members of the first nucleic acid population.

3. The method of claim 1, comprising detecting the at least one target nucleic acid sequence in a sample comprising members of the first or a second nucleic acid population comprising the at least one target nucleic acid sequence, the method comprising the steps of:
providing a set of differentiating nucleic acid probes corresponding to the set of short shared nucleotide sequences;
hybridizing at least one of the differentiating nucleic acid probes to the sample comprising the target nucleic acid and other members of the first or the second nucleic acid population; and,
detecting hybridization of the at least one differentiating nucleic acid probe to the target nucleic acid; and, optionally:
repeating the hybridizing and detecting steps until a plurality of the differentiating nucleic acid probes have been hybridized to the first or the second nucleic acid population and the differentiating nucleic acid probes detected; and, optionally:
determining sets of the differentiating nucleic acid probes that hybridized to the target nucleic acid; or, alternately,
determining sets of the differentiating nucleic acid probes that hybridized to each member of the first or the second nucleic acid population.

4. The method of claim 3, wherein the set of short shared nucleotide sequences is selected from: a nucleic acid sequence database and an expressed sequence tag database; or,
wherein the set of short shared nucleotide sequences corresponds to nucleic acid sequences selected from one or more of: restriction sites, homopolymers, and sequence repeats; or,
wherein the set of short shared nucleotide sequences comprises a set of degenerate oligonucleotides, wherein each degenerate oligonucleotide comprises at least one region of sequence identity and at least one region of sequence heterogeneity as compared to the other degenerate oligonucleotides of the set of degenerate oligonucleotides.

5. The method of claim 3, wherein the first or the second nucleic acid population is selected from: RNAs, DNAs, and cDNAs; or,
wherein the first or the second nucleic acid population is derived from one or more of: animals, plants, fungi, protists, and monera; or,
wherein the first or the second nucleic acid population is derived from a plant selected from the genera: Fragaria, Lotus, Medicago, Onobrychis, Trifolium, Trigonella, Vigna, Citrus, Linum, Geranium, Manihot, Daucus, Brassica, Raphanus, Sinapis, Atropa, Capsicum, Datura, Arabidopsis, Hyoscyamus, Lycopersicon, Nicotiana, Solanum, Petunia, Digitalis, Majorana, Cichorium, Helianthus, Lactuca, Bromus, Asparagus, Antirrhinum, Heterocallis, Nemesia, Pelargonium, Panicum, Pennisetum, Ranunculus, Senecio, Salpiglossis, Cucumis, Browaalia, Lolium, Zea, Triticum, Sorghum, Malus, Gossypium, Vicia, Lathyrus, Lupinus, Pachyrhizus, Wisteria, and Stizolobium; or,
wherein the first or the second nucleic acid population is derived from a crop plant selected from the genera: Agrostis, Phleum, Dactylis, Sorghum, Setaria, Zea, Oryza, Triticum, Secale, Avena, Hordeum, Saccharum, Poa, Festuca, Stenotaphrum, Cynodon, Coix, Olyreae, Phareae, Glycine, Pisum, Cicer, Phaseolus, Lens, and Arachis; or,
wherein the first or the second nucleic acid population is derived from a plant selected from: corn, rice, cotton, soybean, sorghum, wheat, oats, barley, millet, sunflower, rapeseed, canola, peas, beans, lentils, peanuts, yam beans, cowpeas, velvet beans, clover, alfalfa, lupine, vetch, lotus, sweet clover, wisteria, and sweetpea; or,
wherein the members of the first or the second nucleic acid population comprise one or more of: expressed sequence tags, promoters, enhancers, exons, introns, domains, genes, polymorphisms, operons, gene clusters, gene families, and cloned nucleic acids.

6. The method of claim 3, wherein the differentiating nucleic acid probes are polynucleotides comprising about six nucleotides; or,
wherein the differentiating nucleic acid probes are polynucleotides comprising about eight nucleotides; or,
wherein the differentiating nucleic acid probes are polynucleotides comprising about ten nucleotides; or,
wherein the differentiating nucleic acid probes are polynucleotides comprising about twelve nucleotides.

7. The set of short shared nucleotide sequences made by the method of claim 1.

8. The method of claim 1, further comprising providing a set of nucleic acid probes corresponding to the selected set of short shared nucleotide sequences.

9. The set of nucleic acid probes made by the method of claim 8.

10. The method of claim 3, wherein the sample comprises an array of nucleic acids comprising members of the first or the second nucleic acid population; or,
wherein the members of the first or the second nucleic acid population are attached to a solid support; or,
wherein the set of differentiating nucleic acid probes is present in an array of nucleic acids; or,
wherein the differentiating nucleic acid probes are attached to a solid support.

11. The method of claim 3, wherein the sample comprising members of the first or the second nucleic acid population comprises non-standardized concentrations of each member of the first or the second nucleic acid population; or,
wherein the sample comprising members of the first or the second nucleic acid population comprises standardized concentrations of each member of the first or the second nucleic acid population.

12. The method of claim 3, wherein the providing step comprises synthesizing the set of differentiating nucleic acid probes in an automated nucleic acid synthesizer.

13. The method of claim 3, wherein at least one step occurs in vitro or in silico.

14. The method of claim 3, wherein the hybridizing step comprise; concomitantly hybridizing at least one competitor differentiating nucleic acid probe to the differentiating nucleic acid probes, wherein the at least one competitor differentiating nucleic acid probe is complementary to at least one of the differentiating nucleic acid probes, thereby minimizing non-specific cross-hybridization; or,
wherein the target nucleic acid sequence is detected at least twice by identifying members of the first or the second nucleic acid population that hybridize the same set of differentiating nucleic acid probes; or,
wherein the target nucleic acid sequence is detected at least twice by identifying members of the first or the second nucleic acid population that hybridize to the same set of differentiating nucleic acid probes, wherein the nucleic acid sequence comprises a cDNA.

15. The method of claim 3, further comprising detecting at least one polymorphism in at least one member of the first or the second nucleic acid population, wherein the determining steps comprise detecting a hybridization of at least one differentiating nucleic acid probe to the at least one member in addition to the hybridization of the differentiating subset that corresponds to the at least one member, thereby detecting at least one polymorphism in the at least one member of the first or the second nucleic acid population.

16. The method of claim 15, wherein the at least one member of the first or the second nucleic acid population corresponds to a gene or a QTL; or,
wherein the at least one polymorphism which is detected maps the at least one member within the first or the second nucleic acid population.

17. The method of claim 16, wherein the first or the second nucleic acid population corresponds to a multigene family.

18. An integrated system comprising a computer or computer readable medium comprising a database comprising at least one sequence record comprising a plurality of non-homologous character strings corresponding to members of at least one nucleic acid population and at least one derivative sequence record comprising at least one set of short shared nucleotide sequences, the integrated system further comprising a user input interface allowing the user to selectively view the at least one sequence record.

19. The integrated system of claim 18, further comprising one or more components selected from:
a sequence search and selection instruction set which searches the plurality of non-homologous character strings corresponding to the members of the at least one nucleic acid population and selects one or more subsequences common to at least two of the plurality of non-homologous character strings;
an automated nucleic acid synthesizer coupled to an output of the computer or computer readable medium, which automated nucleic acid synthesizer accepts instructions from the computer or computer readable medium, which instructions direct synthesis of at least one set of differentiating nucleic acid probes which corresponds to the one or more subsequences common to the at least two of the plurality of non-homologous character strings;
one or more robotic or microfluidic control elements for manipulating at least one set of differentiating nucleic acid probes or the members of the at least one nucleic acid population, wherein the manipulations are selected from: selecting the at least one set of differentiating nucleic acid probes or the members of the at least one nucleic acid population, reverse-transcribing RNAs, synthesizing the at least one set of differentiating nucleic acid probes, amplifying the members of the at least one nucleic acid population, purifying amplified members of the at least one nucleic acid population, arraying the at least one set of differentiating nucleic acid probes or the members of the at least one nucleic acid population, hybridizing the at least one set of differentiating nucleic acid probes to the members of the at least one nucleic acid population, and flowing the members of the at least one nucleic acid population through at least one channel in a microfluidic device exposed sequentially to at least one labeled set of differentiating nucleic acid probes;
a detector for detecting at least one hybridization pattern corresponding to at least one target nucleic acid sequence;
an instruction set for reverse-transcribing at least one RNA sequence, the at least one RNA sequence comprising an RNA sequence selected from the members of the at least one nucleic acid population, into at least one cDNA sequence;
a user readable output element which displays the one or more subsequences common to the at least two of the plurality of non-homologous character strings produced by the sequence search and selection instruction set; and,
a user readable output element which displays at least one hybridization pattern corresponding to the at least one target nucleic acid sequence.

20. The integrated system of claim 18, the computer or computer readable medium further comprising an instruction set for reverse-transcribing at least one RNA sequence, the at least one RNA sequence comprising an RNA sequence selected from the members of the at least one nucleic acid population, into at least one cDNA sequence, wherein an instruction set selects at least one short shared nucleotide sequence from amongst the members of the at least one nucleic acid population by applying a sequence search and selection instruction set which searches the plurality of non-homologous character strings corresponding to the members of the at least one nucleic acid population and selects one or more subsequences common to at least two of the plurality of non-homologous character strings.

21. A composition comprising one or more libraries selected from:
a library of differentiating nucleic acid probes corresponding to at least one set of short shared nucleotide sequences, wherein the at least one set of short shared nucleotide sequences collectively comprises a plurality of differentiating nucleic acid probe member types, wherein differentiating subsets of the plurality of differentiating nucleic acid probe member types differentiate individual members of a nucleic acid population from each other, and
a library of competitor differentiating nucleic acid probes corresponding to at least one set of nucleic acid sequences that are complementary to at least one set of short shared nucleotide sequences.


SEQUENCE LISTING (2 sequences)

SEQ ID NO: 1 - protein, 33 aa, Artificial Sequence
  Description of Artificial Sequence Consensus initiation sequence
MSSSSSMSSSSSMSRYMRCSATGGCGRSSRYSR

SEQ ID NO: 2 - protein, 23 aa, Artificial Sequence
  Description of Artificial Sequence Consensus termination sequence
SRMSRMSKMSTAARSRMYYMRWS